(19) United States
(12) Patent Application Publication
(10) Pub. No.: US 2010/0083453 A1
(43) Pub. Date: Apr. 8, 2010

(54) DISPOSABLE SANITARY HAND COVER

(76) Inventor: Zahida Abbas, Rochdale (GB)

Correspondence Address: EGBERT LAW OFFICES, 412 MAIN STREET, 7TH FLOOR, HOUSTON, TX, 77002, US

(21) Appl. No.: 12/525,457

(22) PCT Filed: Feb. 7, 2008
(86) PCT No.: PCT/GB08/00413

§ 371 Date: Oct. 14, 2009

(30) Foreign Application Priority Data
Feb. 9, 2007 (GB) 0702480.5

(51) Int. Cl. A47K 7/02; A41D 19/01; A61F 13/15

(52) U.S. Cl. 15/210.1; 2/158; 604/374; 15/227

(57) ABSTRACT

A disposable sanitary hand cover includes a hand covering and a separable outer layer. At least a portion of the outer layer is suitable for impregnation with a sanitary or medical preparation. The outer layer of the hand cover is used first to cleanse an appropriate surface, for example a lavatory seat or similar. The outer layer can then be detached and disposed of to leave the hand covering that can be used to wash and/or dry appropriate areas of the body. The hand covering can then also be disposed of. Preferably, therefore, at least the hand covering is made of a biodegradable material.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to a disposable sanitary hand cover. The hand cover has been designed primarily for use in the ritual washing of the body but also has wider sanitary and medical application uses.

2. Description of Related Art Including Information Disclosed Under 37 CFR 1.97 and 37 CFR 1.98.

GB2417497 describes a personal washing apparatus that can be fitted in lavatories and washrooms to extend the washing facilities available. This is particularly useful to those who require ritual washing facilities at various times of the day prior to prayers and other religious devotions. The apparatus could also be installed in a conventional lavatory cubicle to provide a bidet-type facility.

The object of the invention is to provide a disposable sanitary hand cover that can be used when using the aforementioned apparatus. It is, however, envisaged that the hand cover may have wider application for more general sanitary and medical uses.

### BRIEF SUMMARY OF THE INVENTION

According to the present invention, there is provided a disposable sanitary hand cover comprising a hand covering and a separable, outer layer superimposed over at least part of the hand covering, at least a portion of the outer layer being suitable for impregnation with a sanitary or medical preparation.

It is intended that once a user has donned the hand cover, the outer layer will be used first for cleansing a lavatory seat, bidet or other appliance that may come into contact with the body and, after removal of the outer layer from the hand covering, the latter can be used to wash and/or dry appropriate areas of the body, in particular those areas washed during ritual cleansing. For this purpose, the hand cover can be supplied with the outer layer already impregnated with a suitable sanitary or medical preparation but it will be appreciated that it can also be supplied ready for the user to apply a suitable preparation to the outer layer.

Preferably, therefore, the hand cover comprises an impermeable layer located between at least said portion of the outer layer and the hand covering. This impermeable layer may comprise a soluble or biodegradable plastics film that, at least for a temporary period, prevents water and other liquids from wetting the hand covering when the outer layer has been impregnated with the sanitary or medical preparation. Such a film may comprise a separate layer between the outer layer and the hand covering, which is adapted to be removed from the hand covering along with the outer layer, or it may comprise a coating over the inner side of the outer layer.

Preferably also, the hand cover is in the form of a mitten. Advantageously, the mitten is adapted for use on a left hand in order to satisfy religious practice. However, the mitten can be made universal and suitable for use on either hand

Preferably also, in a first embodiment the outer layer is superimposed over the whole of the hand covering. However, in other embodiments the outer layer is provided on one side of the hand covering. In one of these embodiments the hand cover preferably comprises three layers of material which are secured together along a seam around the periphery of the hand cover, one outer layer and the middle layer of material defining the hand covering and the other outer layer of material forming the separable outer layer. In other of these embodiments, the outer layer comprises a pad that is secured on one side of the hand covering.

Further preferred but non-essential features of the invention are described in the dependent claims.

### BRIEF DESCRIPTION OF THE SEVERAL VIEWS OF THE DRAWINGS

Various embodiments of the present invention will now be described by way of example with reference to the accompanying drawings.

FIG. 1 is a perspective side view of a first embodiment of disposable sanitary hand cover in accordance with the invention.

FIG. 2 is a perspective side view of an inner layer of the hand cover shown in FIG. 1 after an outer layer has been removed therefrom.

FIG. 3 is a cross-sectional view of a modified outer layer of the hand cover along a line III-III located as shown in FIG. 1.

FIG. 4 is a perspective side view of a second embodiment of disposable sanitary hand cover in accordance with the invention.

FIG. 5 is a cross-sectional view along line V-V in FIG. 4.

FIG. 6 is a perspective side view of a third embodiment of disposable sanitary hand cover in accordance with the invention.

FIG. 7 is a perspective side view of a fourth embodiment of disposable sanitary hand cover in accordance with the invention.

### DETAILED DESCRIPTION OF THE INVENTION

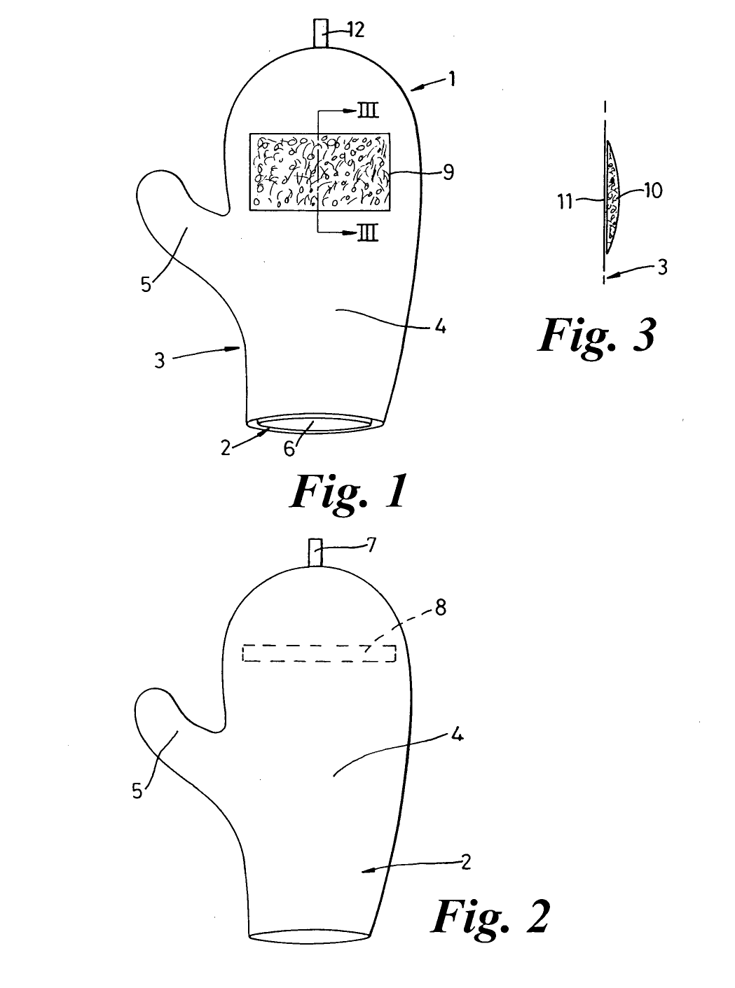

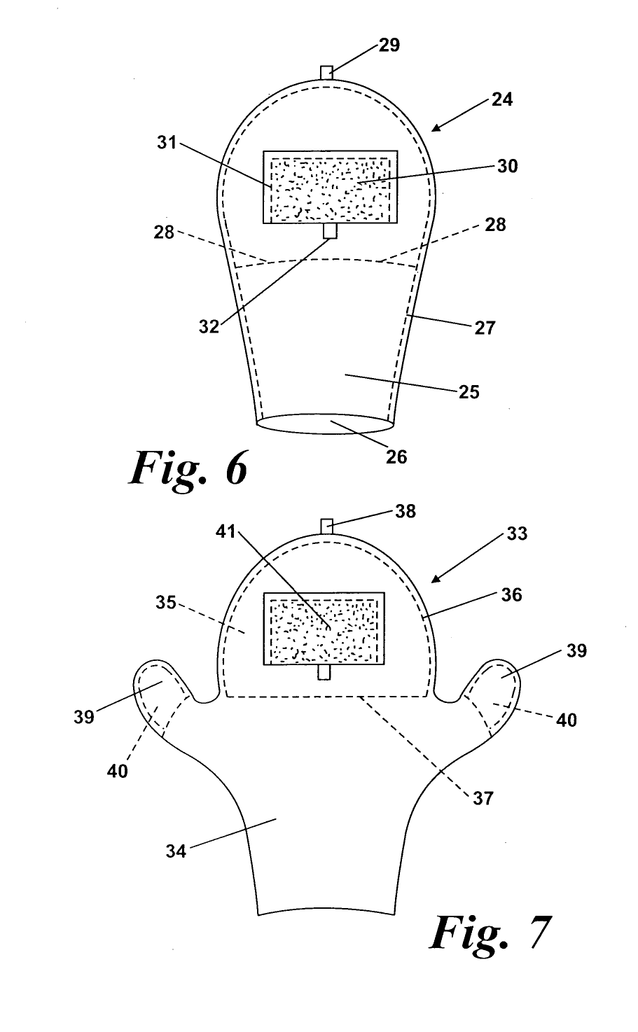

A first embodiment of hand cover 1 according to the invention is shown in FIGS. 1 to 3. In this embodiment, the hand cover 1 is in the form of a mitten with separate, inner and outer layers 2 and 3 respectively. Each layer 2, 3 has a section 4 for the fingers and a section 5 for the thumb. As the cover 1 is primarily to be used for ritual washing, it has been made suitable for use by the left hand It will be appreciated, however, that right-handed or universal mittens, as described below with particular reference to FIGS. 6 and 7, could readily be made for other forms of washing or cleansing. In other embodiments, the hand cover could be made in the shape of a glove with individual finger portions.

The cover 1 is preferably made from biodegradable material, for example using a non-woven biodegradable material such as cellulose, viscose or cotton. Such non-woven materials are relatively inexpensive to produce yet have the strength required for the purpose of the cover. The layers 2 and 3 may be manufactured individually with the outer layer 3 then superimposed over the inner layer 2 to form the cover 1. This means that in use the outer layer 5 can be readily pulled off the inner layer, as described below. Each of the layers 2 and 3 can be manufactured from the aforementioned biodegradable materials by cutting out back and front sides with a mitten-shaped profile and fastening these sides together by stitching, gluing or welding around the edges of the mitten, leaving an appropriate aperture 6 for insertion of the hand A preferred method of fastening the sides of the layers together is to glue them together using industrial starch as an adhesive.

It will be appreciated that the layers 2 and 3 may each be made of materials with different properties. Also, the front side of each of the layers 2, 3, that is the side that in use will cover the palm side of the hand, may be made from a different material to that used for the back side.

The inner layer 2, as shown in FIG. 2, is preferably made of an absorbent material and is provided with an exterior tag 7 so that it can be pulled off the hand with minimal contact to the finger section 2 of the layer 2. In addition, an internal tag 8 is provided that can be gripped or secured around one or more fingers, or in another embodiment the thumb. This tag 8 can then be used to hold the inner layer 2 in position covering a hand of the user while the outer layer 3 is removed therefrom.

The outer layer 3, as shown in FIG. 1, comprises a portion 9 that is suitable for impregnation with a sanitary or medical preparation. This portion 9 is made from an absorbent material that retains the preparation and that, when pressed by a user against a surface, such as lavatory seat, bidet or other appliance that may come into contact with the body, releases the sanitary preparation to cleanse the appliance. It will be appreciated that in other uses of the hand cover, for example a medical use, the portion 9 may be used to cleanse other surfaces, such as instruments or even the skin of a patient. Preferably, therefore, as shown in FIG. 1, the portion 9 is located in the finger section 4 of the hand cover 1 that will lie adjacent the ends of a user's fingers.

In order that the portion 9 can retain the sanitary or medical preparation, the front side of the layer 3 may be made from an absorbent material and the portion 9 comprises a predefined area of this side into which the sanitary preparation is impregnated. In this case the outer layer 3 is preferably made of a material which is provided with a coating on one side, such as a plastics coating, that forms an impermeable film. In the finished cover, this film is located on the inner side of the layer 3 and prevents the sanitary preparation from seeping through the back of the portion 9 and wetting the inner layer 2.

In a modification, as shown in particular in FIG. 3, a spongy or absorbent pad 10 is impregnated with a sanitary or medical preparation and is then adhered to an underlying portion 11 of the layer 3. Such a pad 10 may not be biodegradable but is preferably detachable so that it can be disposed of separately. In addition, the inner surface of the pad 10 is preferably made of an impermeable material such as a plastics film to prevent the preparation from wetting the rest of the hand cover 1. It will be appreciated that in this embodiment there is no requirement for the layer 3 to be made absorbent and it could be made from an inexpensive material such as paper to offset the cost of producing the pad. In another embodiment, the pad 10 made be adhered directly to the inner layer 2 provided it can be satisfactorily detached for disposal and leave the inner layer 2 in a condition suitable for washing or drying the body.

However, in the embodiment as shown in FIGS. 1 and 2, the outer layer 3 is also provided with an exterior tag 12 so that it can be pulled off the hand, leaving the inner layer 2 in place, with minimal contact to the finger section 4 of the layer 2, in particular the portion 9.

It will also be appreciated that although the embodiment shown in FIGS. 1 and 2 is handed, the hand cover 1 could be readily made universal and suitable for use on either hand by the provision of a portion 9, as described above, on both sides of the outer layer 3.

In use, a user should don the hand cover 1 prior to ritual cleansing or personal washing. The user can then press the impregnated portion 9 or the pad 10 against a surface to release the sanitary or medical preparation that may then be rubbed over the surface to cleanse or treat it as appropriate. Once this has been done, the user can discard the outer layer 3 of the cover 1 by pulling it off his hand with the other hand, using the tag 12. In order to retain the inner layer 2 over the hand, the tag 8 inside the layer 2 can be gripped. Thereafter, the absorbent inner layer 2 of the hand cover 1 can be used to wash and/or dry appropriate areas of the treated surface, as required. Once this has been done, the inner layer 2 can be pulled off the hand using the tag 12 and also discarded. Preferably, the biodegradable nature of the hand cover 1 is such that it can be flushed away down a lavatory after use but this may not be appropriate in all cases.

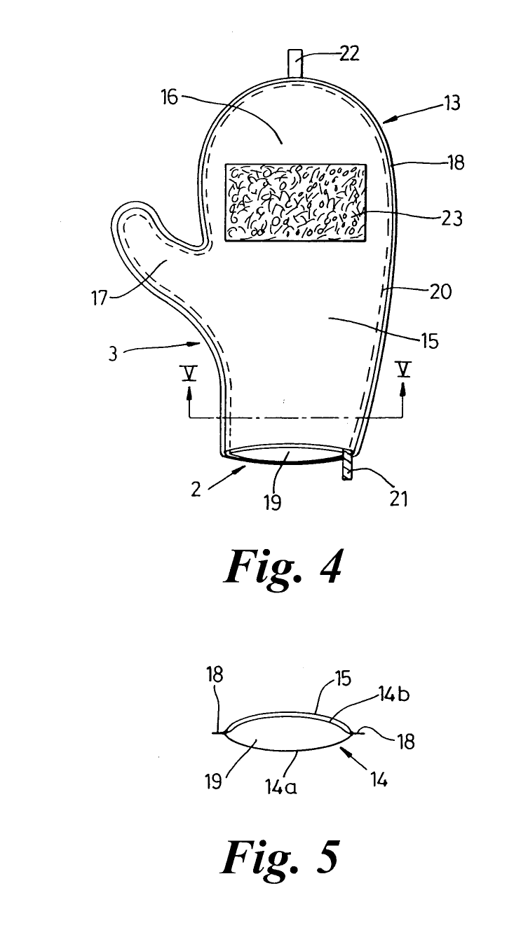

A second embodiment of hand cover 13 according to the invention is shown in FIGS. 4 and 5. In this embodiment, the hand cover 13 is also in the form of a mitten with a hand covering 14 and a separable outer layer 15 that covers at least a substantial part of one side of the covering 14. As in the first embodiment, the hand covering 14 has a section 16 for the fingers and a section 17 for the thumb. Preferably, the outer layer 15 covers at least the finger section 16 of the palm-side of a left-handed hand covering 14. The hand cover 13 is again made from a biodegradable material, as described above, and preferably from one which permits the outer layer 15 to be readily separated, via, from a residual part of itself. In practice, therefore, at least the outer layer 15 is likely to be made from a cellulose-based material or other absorbent, paper-like material.

The hand cover 13 is preferably made from three layers 14a, 14b, 15, each cut into a mitten-shaped profile from a suitable material, that have been secured together along a seam 18 formed around the periphery of hand cover 13. The layers 14a, 14b, 15 can be secured together by stitching, gluing or welding, leaving an appropriate aperture 19 for insertion of the hand. As before, the layers are preferably adhered together using an industrial starch. Also, as in the first embodiment, the layers 14a, 14b, 15 may each be made of materials with different properties and the layers 14b and 15 that, in use, will cover the palm side of the hand, may be made from a different material to the layer 14a used for back side of the cover 13. The outer layer 14a and the middle layer 14b together form the mitten-like hand covering 14 whereas the layer 15 forms the separable outer layer. To this end, the layer 15 is preferably provided with a line of perforations 20 around its periphery adjacent the seam 18 in order that it can be torn away from the hand covering 14. In addition, the inner side of the layer 15 is preferably coated or covered with an impermeable layer that, at least for a temporary period, prevents water and other liquids from wetting the layers 14a, 14b when the outer layer has been impregnated with the sanitary or medical preparation. Preferably, the impermeable layer comprises a soluble or biodegradable plastics coating or film that is removed from the cover 13 along with the layer 15. At least one tag 21 is provided adjacent the aperture 19 at the wrist portion of the mitten to assist in removal of the layer 15. In addition, an external tag 22 may be provided for removal of the hand covering 14 at top of the finger section 16.

As in the first embodiment, the outer layer 15 comprises at least a portion 23, preferably located in the finger section 16 of the hand cover 13, that is adapted to be impregnated with a sanitary or medical preparation. This can be done by printing a suitable preparation onto the layer 15 prior to it being incorporated into the hand covering. In another embodiment, this portion 23 may comprise a spongy or absorbent pad that is impregnated with a sanitary or medical preparation and secured to the outer layer 15 in a similar fashion to the arrangement described above with reference to FIG. 3. In yet another embodiment, the hand cover 13 is supplied without having been impregnated with any preparation, it being left to the use to apply such a preparation thereto immediately prior to use. To this end, the whole of the layer 15 may be adapted to be impregnated with such a preparation rather than a particular area.

The use of hand cover 13 is similar to that of the hand cover 1. After donning it, the user can use the impregnated portion 23 of the layer 15 as required. If the hand cover 13 is not already impregnated with any preparation, then one can be applied thereto, for example by spraying, immediately prior to use. Once the layer 15 has been used, it can be separated from the hand covering 14 by tearing it away along the line of perforations 20 using the tag 21. Thereafter, the hand covering 14 made of the layer 14a, 14b can be used to wash and/or dry appropriate areas, for example of the body. Once this has been done, the hand covering 14 can be removed using the tag 22 and disposed of.

A third embodiment of hand cover 24 according to the invention is shown in FIG. 6. In this embodiment, the hand cover 24 is also in the form of a universal mitten without a thumb portion and can therefore be located over either hand As previously described, the mitten 24 is made from two layers of material 25, 26 that are secured together along a seam 27 formed around the periphery of hand cover 24, leaving an appropriate aperture for insertion of the hand. In a modification, the layer 26 of the mitten that covers the back of the hand need only cover the fingers and can therefore terminate at a higher position, as shown by a dashed line 28, than the layer 25 on the front side of the mitten 24. In this modification, the material from which at least the layer 25 is made preferably has some rigidity so that the area covering the palm of the hand, which is unsupported by the rear layer 26, remains in place and does not flop down over the portion covering the fingers when in use. This material is again preferably a cellulose-based material or other absorbent, paper-like material that is biodegradable. At the top of the hand cover 24, a tab 29 can be provided so that it can be readily pulled off the hand after use.

As in the first embodiment described above, a portion of the hand cover 24 is adapted to be impregnated with a sanitary or medical preparation. This portion preferably comprises a spongy or otherwise absorbent pad 30 that is secured, for example by an adhesive, to the front layer 25 around its periphery, for example around three sides if rectangular as in the illustrated embodiment. However, a line of perforations 31 in the structure of the pad 30 adjacent its line of securement enables it to be detached from the hand cover 24 and a tab 32 is provided on the pad 30 so that it can be readily pulled away from the hand cover 24 after use. The inner surface of the pad 30 that contacts the layer 25 is made of an impermeable material such as a plastics film to prevent wetting of the layer 25 when the pad is impregnated with the sanitary preparation. As previously, it is envisaged that the pad 30 can be either pre-impregnated with an appropriate preparation or the hand cover 24 can be supplied without impregnation, it being left to the user to apply such a preparation to the pad 29 immediately prior to use.

A fourth embodiment of hand cover 33 is shown in FIG. 7. This is similar to the embodiment shown in FIG. 6 insofar as it comprises two layers of material 34, 35 that are secured together along a seam 36 formed around the periphery of hand cover 33 and with the layer 35 for covering the back of the hand 24 terminating at a higher position, as shown by a dashed line 37, than the layer 34 for covering the front of the hand A tab 38 is provided at the top of the hand cover 33 to assist in its removal after use. However, this hand cover 33 is made universal by the provision of areas 39 for covering the thumb on both side edges of the layer 34. These areas 39 are made into a sleeve that can be attached to the thumb by the provision of small pieces of material 40 that are secured thereto similar to the back of the layer 35 although they do not necessarily comprise part of the layer 35. A pad 41 is secured to the layer 34 in the same manner as described above with reference to FIG. 6. In use, this hand cover 33 is used in the same way as before but only the relevant thumb covering is used, the other can be left empty and has no effect on the use of the hand cover 33.

In all of the embodiments described above, when the preparation is pre-impregnated into the hand cover it preferably comprises a proprietary preparation such as one suitable for cleansing or disinfecting surfaces that is non-irritant to the skin Also, it is preferably non-volatile so that the hand cover 1, 13, 24, 33 has a reasonable shelf-life and does not need to be individually wrapped.

It is expected that a plurality of hand covers 1, 13, 24, 33 in accordance with the invention could be provided in a receptacle close to a personal washing apparatus as mentioned above, possibly in conjunction with appropriate dispensers for the sanitary preparation and bin facilities for collecting used covers 1, 13, 24, 33 for disposal. However, preferably, the hand covers are made of completely soluble materials in order that they can be flushed down a lavatory after use. Similarly, the packaging for such a hand cover is also preferably made from biodegradable materials.

Although the hand cover 1, 13, 24, 33 has been designed with a view to it being used primarily for ritual washing for religious purposes, it will be appreciated that it could be used in many other ways. For example, it could be used for general household or industrial cleaning and in addition be used for cosmetic purposes or personal cleansing unconnected with religious practice. It may also have application within the medical sphere for use in various medical or surgical procedures. In this case, the hand cover can be supplied in a sterile state and individually wrapped in a sterile packaging for use as required.

1. A disposable sanitary hand cover comprising:
a hand covering; and
a separable, outer layer superimposed over at least part of the hand covering, at least a portion of the outer layer being suitable for impregnation with a sanitary or medical preparation.

2. A hand cover as claimed in claim 1, further comprising:
an impermeable layer located between at least said portion of the outer layer and the hand covering.

3. A hand cover as claimed in claim 2, wherein the impermeable layer comprises a plastic film removable from the hand covering along with the outer layer.

4. A hand cover as claimed in claim 2, wherein the impermeable layer comprises a coating on the inner side of the outer layer.

5. A hand cover as claimed in claim 1, wherein the outer layer is superimposed over and covers the hand covering.

6. A hand cover as claimed in claim 1, wherein the outer layer is provided only on one side of the hand covering.

7. A hand cover as claimed in claim 6, further comprising:
three layers of material secured together along a seam around the periphery of the hand cover, having one outer layer and a middle layer of material defining the hand covering and the other outer layer of material forming the separable outer layer.

8. A hand cover as claimed in any of any of claim 1, wherein the outer layer comprises a pad secured on one side of the hand covering.

9. A hand cover as claimed in claim 6, wherein the outer layer comprises a line of perforations around a periphery thereof and being tearable away from the hand covering.

10. A hand cover as claimed in claim 1, wherein the hand covering is in the form of a mitten.

11. A hand cover as claimed in claim 1, wherein said portion is located in an area of the hand cover, adjacent the ends inserted into the cover.

12. A hand cover as claimed in claim 1, wherein said portion of the outer layer is comprised of an absorbent material, retaining sanitary or medical preparation and releasing the preparation when pressed against a surface.

13. A hand cover as claimed in claim 12, wherein said absorbent material comprises a pad secured to an underlying portion of the outer layer.

14. A hand cover as claimed in claim 12, wherein at least part of the outer layer is comprised of an absorbent material, said portion of the outer layer comprising a predefined area of said part of the layer impregnated with the sanitary or medical preparation.

15. A hand cover as claimed in claim 1, wherein the outer layer is provided with a tag to facilitate its separation from the hand covering.

16. A hand claimed in claim 1, wherein the hand covering is comprised of an external tag to facilitate removal after use.

17. A hand cover as claimed in claim 1, wherein the hand covering is comprised of an internal tag gripped or secured around a digit whereby in use the hand covering can be retained in position while the outer layer is separated therefrom.

18. A hand cover as claimed in claim 1, wherein at least one side of the hand covering is comprised of an absorbent material.

19. A hand cover as claimed in claim 1, primarily being comprised of a non-woven, cellulose-based material.

20. A hand cover as claimed in claim 1, wherein the hand covering is comprised of a biodegradable material.

21. A hand cover as claimed in claim 1, wherein the outer layer is comprised of a biodegradable material.

22. A hand cover as claimed in claim 1, being adapted for use on either a left or a right hand.
